(19) United States
(12) Patent Application Publication
(10) Pub. No.: US 2022/0061823 A1
(43) Pub. Date: Mar. 3, 2022

(54) METHOD FOR OBTAINING EXHALED RESPIRATORY SPECIMENS

(71) Applicant: Aeolus Partners, LLC, New Bedford, MA (US)

(72) Inventors: Barry M. Berger, Brockton, MA (US); Charlotte Kuperwasser, Newton, MA (US)

(21) Appl. No.: 17/461,698

(22) Filed: Aug. 30, 2021

Related U.S. Application Data
(60) Provisional application No. 63/072,828, filed on Aug. 31, 2020.

(51) Int. Cl. A61B 10/00; A41D 13/11; G01N 1/22; G01N 33/497

(52) U.S. Cl. CPC A61B 10/0051; A41D 13/11; G01N 2001/2244; G01N 33/497; A61B 2010/0087; G01N 1/2214

(57) ABSTRACT

The invention provides articles, devices, and methods for sampling respiratory droplets, the article comprising a material capable of adsorbing or absorbing respiratory droplets such that the contents of said respiratory droplets can be recovered for analysis. The invention allows respiratory droplets to be collected directly from any source of moisture and commonly-used devices, for example face masks and air handling systems.

### TECHNICAL FIELD

This disclosure relates to articles and methods for obtaining exhaled respiratory specimens for the purpose of diagnostic testing for infectious agents.

### BACKGROUND

Global responses to droplet-spread viruses, such as the influenza virus, have required tracking and tracing programs that require consistent diagnostic testing of individuals for the infectious agent and tracing locations and individuals that have been exposed to infection. The diagnostic tests require, as inputs for analysis, biological specimens sampled from healthy individuals and patients, who may or may not be symptomatic, and may or may not be infected. Tests for respiratory infections are commonly performed on saliva, nasopharyngeal, oropharyngeal, nasal mid-turbinate, and anterior nares specimens. These specimens are most frequently collected by a range of invasive sampling methods, which include swabbing nasal and oral passages with flocked, spun polyester, or tapered swabs, and saline washes using bulbs, syringes, or vacuum-assisted aspiration. Diagnostic tests can also be performed on specimens extracted from the lower respiratory tract, which can be sampled through the spontaneous or induced production of sputum, aspiration, or bronchoalveolar lavage.

These collection methods, however, require that individuals either visit a clinician or be sent specialized equipment for collecting samples. As a result, the frequency at which individuals are tested is limited by the availability of clinicians and equipment. Testing aerosols bearing infectious agents from ambient circulating air has proven even more difficult, requiring expensive and complicated machinery that involves cooling air to collect liquid condensate. Moreover, many machines used to draw pathogens out of air end up shredding the pathogens they are meant to capture, rendering the pathogens unrecoverable for analysis. Without simple, frequent and readily available testing of individuals and locations, a virus can continue to spread through a population, threatening to overload local medical systems, and resulting in thousands or hundreds of thousands of preventable deaths.

### SUMMARY

The present invention provides articles, devices, and methods for collecting respiratory droplets directly from any source of respiratory droplets and commonly-used devices, for example face masks and air handling systems. As a result, respiratory droplets and their contents may be collected from individuals during their day-to-day activities without the need for a clinical visit or without the need for expensive or complicated at-home tests. The invention provides a layer containing a material capable of adsorbing or absorbing respiratory droplets. For example, the material may be a hygroscopic layer (that may be removable) of a facemask or a layer on an air filtration system that, in either case captures exhaled droplets, are contemplated by the invention. The common feature of articles and devices of the invention is the use of a material, such as a hygroscopic material, to collect droplets from any source in a manner that allows the droplets to be recovered from the material and analyzed for the presence of an infectious agent or its associated proteins or nucleic acids. In the case of a face mask, the source is moisture and respiratory droplets from the nose or mouth. In the case of other systems, such as HVAC, air handlers, filters and the like, the sources are aerosolized droplets trapped from the environment. To facilitate analysis, the article or collection portion of a device of the invention preferably comprises a hygroscopic material that is capable of adsorbing or absorbing moisture from any source, such that the contents of the respiratory droplets can be readily recovered. For example, when using an adsorbing material, an aqueous buffer can be used to elute the droplets from the article to recover the droplets for analysis.

Articles of the invention may be included in or configured to be used as collection devices and day-to-day use devices. For example, the invention may comprise a hygroscopic layer, which may be a removable insert of a face mask. Exhaled viral particles are captured by the layer, which may then be submitted for analysis. The face mask may be a surgical mask, such as an N95 surgical mask. Traditionally, the materials used in surgical masks would not normally adsorb or absorb droplets that could be later tested. This is because droplets cannot be collected from the material of surgical masks in the quantities and at a quality that would allow the droplets to be tested. By the present invention, comprising an article that is a removable insert, testing can easily be accomplished by removing the insert and submitting it for testing. Advantageously, the article has the ability to accumulate respiratory droplets and their contents from any source for the entire duration of time that the article is employed, leading to increased sampling of exhaled infectious particles over time. This both increases the viral (infectious agent) load being tested and also allows for sampling of infectious agents over a period of time, rather than at only a single point in time. Infected individuals can then be easily identified, and viral infections can be tracked and traced and the quantity of infectious agent released over time calculated which may further inform risk of spread of infection.

A key feature of the invention is adsorbent or absorbent adherence, using any known material, for example a hygroscopic material, of respiratory droplets from any source. The respiratory droplets may comprise viral particles, and may be collected proximal to a point at which the droplets would typically be dispersed in the environment. Thus, a device of the invention may be a face mask or a filter in an air distribution system, dehumidifier, fan and the like. In the context of a face mask, the invention comprises an outer layer and an inner layer comprising an absorbent or adsorbent material capable of collecting respiratory droplets such that the contents of said droplets can be recovered for analysis. The droplets recovered for the analysis can be analyzed for the presence and/or identify of viral particles.

Advantageously, the material used in the article of the invention can be any material capable of adsorbing or absorbing respiratory droplets. For example, the material may comprise a hygroscopic material, such as a desiccant. The desiccant may be a silica-based desiccant, such as a silica gel. The material may be a superabsorbent polymer, for example a sodium polyacrylate. This contrasts with existing methods for the analysis of air, which collect exhaled condensation through the use of expensive and often cost prohibitive machinery.

The article may also be provided in any available shape suited for the intended use. For example, the article may be shaped as an insert for a face mask. The article may also be shaped to filter circulating air. For example, the article may be shaped as a filter for an HVAC system or for a circulating air vent.

The article of the invention may also be shaped to be included in a collection device. The article of may also be shaped to fit within a collector tube. For example, the collector tube may be a nasal exhalant collector tube. The collector tube may be an oral exhalant collector tube. Advantageously, the article may be shaped as dispersible beads within the collector tube. The collection device may be a hand-held collection device. For example the device may be a lollipop-type device that can be held by the user in front of their face. The user may hold the article up to their face and exhale onto the article. The device may be a tea-bag type device, in which the article is part of a series of layers and exhaled droplets may be collected within those layers. The article may also be shaped as a straw-like collection device or to fit within a straw-like collection device. The article may also be free standing. For example, the article may lay flat on a surface and collect respiratory droplets from ambient or circulated air that comes in contact with the surface.

The invention also provides devices comprising the article of the invention. For example, the invention provides face masks, such as surgical masks, filters for circulating air or ambient air, such as filters for HVAC systems, filters for ventilation systems, and filters for a fans and collection device, such as oral collector tubes, nasal collector tubes, hand-held lollipop devices, hand-held tea-bag devices, surface-resting devices, straw-based devices, or inserts for a straw-based devices, comprising the article of the invention.

The invention also provides for methods of sampling respiratory droplets using articles and devices of the invention.

The invention provides a method for sampling respiratory droplets, the method comprising contacting with respiratory droplets a material capable of adsorbing or absorbing the contents of the respiratory droplets, recovering the contents of the respiratory droplets from the material, and analyzing the recovered contents of the respiratory droplets. The respiratory droplets may be adsorbed or absorbed from one or more of the group consisting of moisture, exhaled air, circulating air, and ambient air. The material capable of adsorbing or absorbing respiratory droplets may be a hygroscopic material. Advantageously the hygroscopic material is capable of adsorbing respiratory droplets and the contents of the respiratory droplets may be recovered by elution. Elution may be accomplished with an aqueous buffer.

### BRIEF DESCRIPTION OF THE DRAWINGS

FIG. 1 depicts an article of the invention shaped for use with a face mask.

FIG. 2 depicts an article of the invention shaped as a face mask.

FIG. 3 depicts articles of the invention for use with face masks.

FIG. 4 depicts articles of the invention for use with face coverings.

FIG. 5 depicts an exemplary facemask using the invention.

FIG. 6 depicts an article of the invention shaped for use with a surgical mask.

FIG. 7 depicts an article of the invention shaped for use with a surgical mask.

FIG. 8 depicts a collector tube of the invention.

FIG. 9 depicts a collector tube of the invention with a mouthpiece.

FIG. 10 depicts a collector tube of the invention with a mouthpiece.

FIG. 11 depicts a cross-section of a collector tube of the invention.

FIG. 12 depicts a collector tube of the invention without a mouthpiece.

FIG. 13 depicts collector tubes of the invention.

FIG. 14 depicts a nasal collector tube of the invention.

FIG. 15 depicts a nasal collector tube of the invention.

FIG. 16 depicts a hand-held collection device of the invention.

FIG. 17 depicts a straw-type collection device of the invention.

FIG. 18 depicts a “tea-bag” device of the invention.

FIG. 19 depicts a “tea-bag” device of the invention.

FIG. 20 depicts a “tea-bag” device of the invention with a surgical mask.

FIG. 21 depicts a “tea-bag” device of the invention with a surgical mask.

FIG. 22 depicts results of an adsorbance titration curve for silica gel beads of the invention.

FIG. 23 depicts results for the saturation of exhaled vapors of beads of the invention.

FIG. 24 depicts results for the recovery of droplets from beads of the invention.

FIG. 25 depicts results for the recovery of droplets from beads of the invention.

FIG. 26 depicts results for the detection of inactivated SARS-CoV-2 virus from silica gel beads of the invention.

### DETAILED DESCRIPTION

The invention provides articles, devices, and methods for sampling respiratory droplets, the article comprising a material capable of adsorbing or absorbing respiratory droplets such that the contents of said respiratory droplets can be recovered. Because the droplets can be adsorbed or absorbed such that the contents of the droplets can be recovered, the contents of the droplets can then be analyzed, for example for the presence of and/or to identify microbes such as viral particles.

Advantageously, the invention has the ability to accumulate respiratory droplets from any source, for example, moisture, such as from saliva or exhaled air, circulating air, and ambient air for any duration of time that the invention is employed, leading to increased sampling of infectious agents over time. This contrasts with the most common sampling methods which only provide samples for infectious agent analysis obtained at a single time point.

Additionally, the articles and methods of the present invention may directly sample droplets from exhaled breath (exhalant) from the respiratory tract, which are the direct biological material responsible for the transmission of respiratory infectious agents between individuals. In contrast, sampling methods that collect different biological material, while possibly containing the infectious agent, only serve as indirect surrogates of the capacity of an individual to infect those in their immediate surroundings and/or the create a broader environmental hazard from aerosolized droplet persistence in the general vicinity. Thus, a key feature and advantage of the present invention is that it provides articles and methods to measure how infectious an individual will be to those in their immediate surroundings.

The length of time over which the invention is applied and/or the length of time that source of droplets to be analyzed is in contact with the article or device of the invention is a variable aspect of the invention, and can be varied to modulate the amount of infectious agent collected so as to accommodate different biologic characteristics of an infectious agent and thereby modulate the sensitivity and specificity of the downstream analytic method used to identify the infectious agent. For example, the sensitivity of a given analytic method can be increased by increasing the amount of time the article is in contact with the source of droplets to be analyzed, leading to increased accumulation of the infectious agent and an opportunity to both detect the presence of the agent and/or to determine the rate and quantity of the infectious agent in the source of the droplets over time. In aspects of the invention, the length of time over which the source of droplets is in contact with the articles or devices of the present invention (for example as incorporated into a naso/oral facial covering, a breath collection tube, or present without being incorporated into a device or specific container) may be varied.

The adsorbent or adsorbent material used in the article or devices of the invention can be any known material capable of collecting respiratory droplets. Advantageously, the material may be a material that collects moisture directly from air and respiratory droplets in the air can collected and the contents of the respiratory droplets can then be recovered, for example by elution, for analysis. For example, the material may comprise silica gel, a cellulose-based absorbent such as corn husk, polymeric foam, sodium polyacrylate, sodium alginate, hyaluronic acid, or other water-absorbing hydrogels.

Silica gel is an amorphous and porous form of silicon dioxide (silica), consisting of an irregular tridimensional framework of alternating silicon and oxygen atoms with nanometer-scale voids and pores. The voids may contain water or some other liquids, or may be filled by gas or vacuum.

A polymeric foam is a foam, in liquid or solidified form, formed from polymers. Examples include: Ethylene-vinyl acetate (EVA) foam, the copolymers of ethylene and vinyl acetate; also referred to as polyethylene-vinyl acetate (PEVA) Low-density polyethylene (LDPE) foam, first grade of polyethylene (PE)

Sodium polyacrylate is a sodium salt of polyacrylic acid with the chemical formula [—CH2-CH(CO2Na)-]n.

Sodium alginate is derived from brown algae or seaweed and forms heat stable gels in the presence of calcium.

The invention may further comprise infectious agent specific molecules, for example antibodies and aptamers that bind to constituents of the infectious particle, including nucleic acids, cell membranes, and intracellular structural proteins and/or intracellular contents, whether from intact or degenerating infectious agent or released from intact or degenerating infectious agent. The invention may comprise hybrid capture probes to bind the target pathogen's DNA or RNA. Proteins, antibodies, and any other known molecule may be used in the article to bind targets. The target may be a viral capsid protein.

Advantageously, the invention may further comprise an indicator of moisture absorption, for example a colorimetric indicator. The colorimetric indicator may comprise any known colorimetric assay, for example the colorimetric indicator may be methyl violet and may be in silica gels, thereby leading the gels to turn from orange, when dry, to green when hydrated. The colorimetric indicator may be provided together with the material capable of adsorbing or absorbing respiratory droplets. The colorimetric indicator may be provided separately from the material capable of adsorbing or absorbing respiratory droplets.

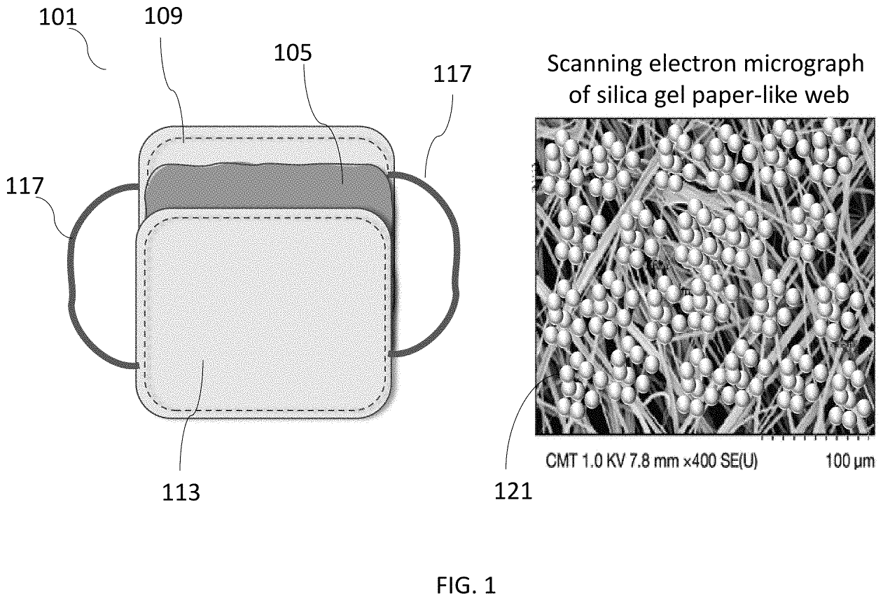

FIG. 1 depicts an exemplary article of the invention. The article 105 is shaped to provide a filter for a facial covering 101, such as a mask, comprising an inner layer 109 and an outer layer 113. The inner and outer layers of the facial covering may comprise a woven or non-woven material. The article comprises a material capable of adsorbing or absorbing respiratory droplets, for example a silica gel comprising a paper-like web 121. In the silica gel 121, incorporated into the gel are a paper-like web of inorganic fibers, such as a desiccant paper, that is placed in between two pieces of fabric secured to one another. The facial covering has ear loops 117 on either side of the facial covering that enable it to be securely attached to cover the nose and mouth region.

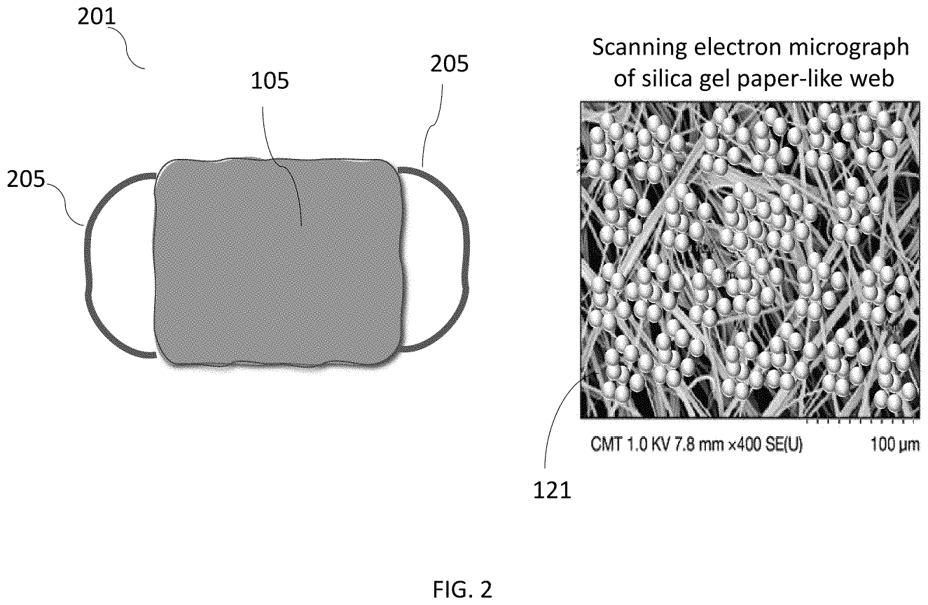

FIG. 2 depicts another exemplary article of the invention. The facial covering 201 comprises only the absorbent or adsorbent material 105, without the inner or outer layers of the facial covering. The adsorbent or adsorbent material itself may be molded to cover the nose and mouth area of the individual. For example, a silica gel incorporating a paper-like web 121 may be molded to conform to cover the nose and mouth area. The facial covering has ear loops 205 on either side of the facial covering that enable it to be securely attached to cover the nose and mouth region.

The article may also be free standing. For example, the article may lay flat on a surface and collect respiratory droplets from ambient or circulating air that comes in contact with the surface. An individual may also hold the article up to their face and exhale into the article. In aspects of the invention, the article may comprise a handle that allows the user to hold the article in place to make contact with exhaled respiratory droplets. The handle may allow the user to hold the article at variable distances and lengths of time from the nose or mouth. The article may comprise a “tea-bag” shape, with the material incorporated into the pouch shape of the tea bag. For example, the “tea-bag” article may comprise food-grade mesh bags, for example nylon tea bags. The “tea-bag” article may comprise a string for the user to hold the article and exhale onto the pouch shape of the tea bag. The “tea-bag” article may be affixed to clothing of the user, for example affixed by a pin and worn like a badge by the user. The “tea-bag” article may comprise colorimetric beads that change color to indicate when the “tea-bag” is saturated with moisture.

The article may come in a package together with instructions that instruct the user as to the distance at which to hold the article in place. The article may come in a package together with instructions that instruct the user as to the length of time for which to hold the article in place. The “tea-bag” article may be shipped for analysis dry, or may be packaged together with an extraction buffer or stabilizing buffer, for example in a sealed vial. The “tea-bag article” may be being placed on the inner surface of a facial covering, for example a surgical mask.

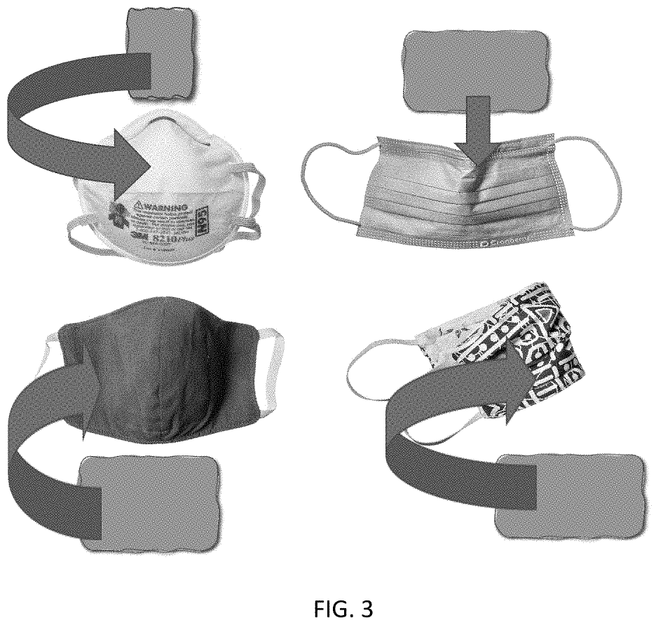

FIG. 3 depicts additional aspects of the article of the invention. The article of the invention can be placed between the inner surface of surgical masks, such as N95 surgical masks, and the face of the user to cover the nose and mouth, irrespective of whether or not the covering method has ear loops. Additionally, the article of the invention may be secured to the facial covering in any known manner. For example, article may be placed between two layers of the facial covering and/or may be fixed to the facial covering by an adhesive. Where the facial covering has a two layered design, the article may be adhered to a fabric layer. Facial covering may also comprise additional layers, for example three layers or more than three-layers.

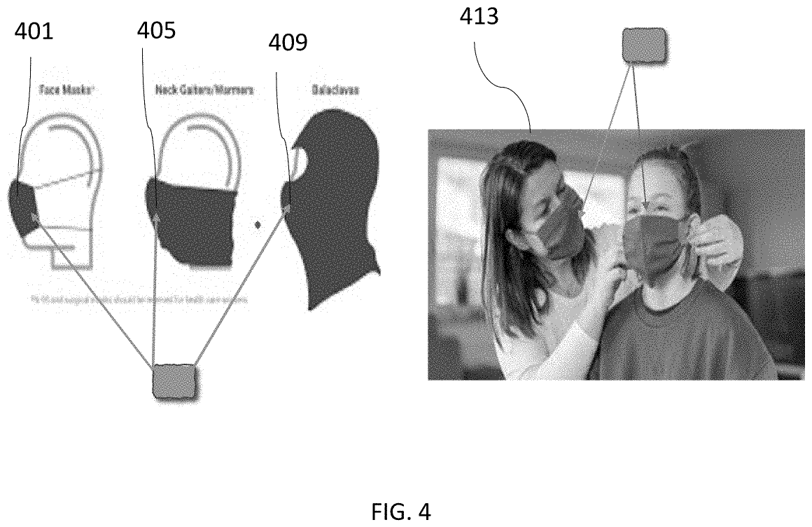

FIG. 4 depicts additional aspects of the article of the invention. The facial covering may have ear loops on either side that enable it to be securely attached to cover the nose and mouth region 401. Facial covering may also exclude ear loops. The facial coverings may be secured to the face of an individual by the individual's hands, through a headband, a scarf, sliding the facial covering into the collar of a shirt or another article, as part of a gator 405 or as a balaclava 409. In aspects of the invention, the facial covering only covers the mouth and not the nose of the individual. Alternatively, the facial covering may covers the nose of the individual and not the mouth of the individual. Advantageously, because the article may be shaped to be included in any variety of facial coverings, the article provides non-invasive collection of respiratory droplets over time from any source, including at the point of contact with exhaled air from the user. This provides an additional advantage in the pediatric setting 413, where individuals may be reluctant to submit to invasive collection methods.

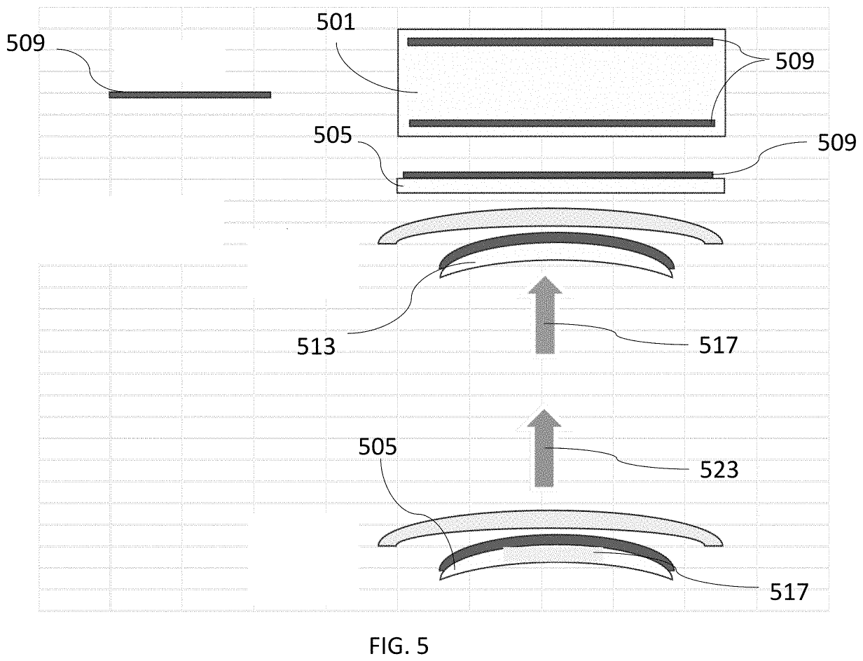

FIG. 5 depicts an aspect of the article of the invention. The material 513, for example a silica gel paper, is part of a facial covering 501. The facial covering may comprise at least one bendable wire 509 that permits the facial covering to better fit the user. The at least one bendable wire may be embedded in the filter. Air, depicted by orange arrows, comprising respiratory droplets comprising microbes 517, depicted as white dots, passes through the article and the respiratory droplets from the air are collected by the material. Along with the respiratory droplets, the microbes are also collected by the material. Because microbes have been collected by the material, air that has passed 523 through the article, comprise fewer microbes. The article may be configured so that air that has passed through the article may be reasonably free of microbes.

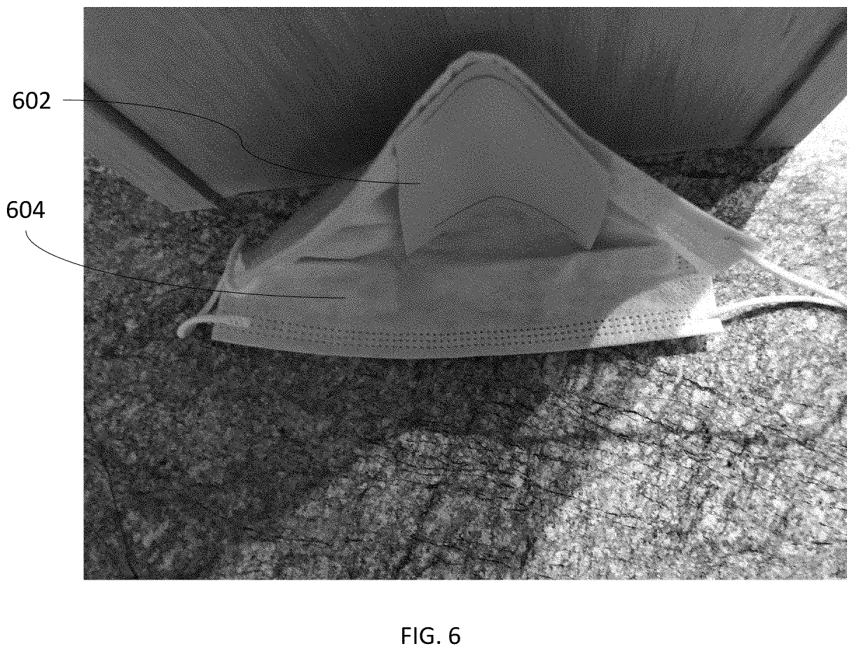

FIG. 6 depicts an aspect of the article of the invention configured for use with a surgical facemask 604. The article of the invention is embedded into the surgical mask 604. The article may be a silica gel sheet 602. Advantageously, the article may be embedded with a wire of custom shape similar to the wire used in the surgical mask 604 in order to allow the article to be curved to match the curvature of the mask. The curvature of the article leaves space from the mouth and the nose of the wearer and decreases contact between the article and the lips of the wearer.

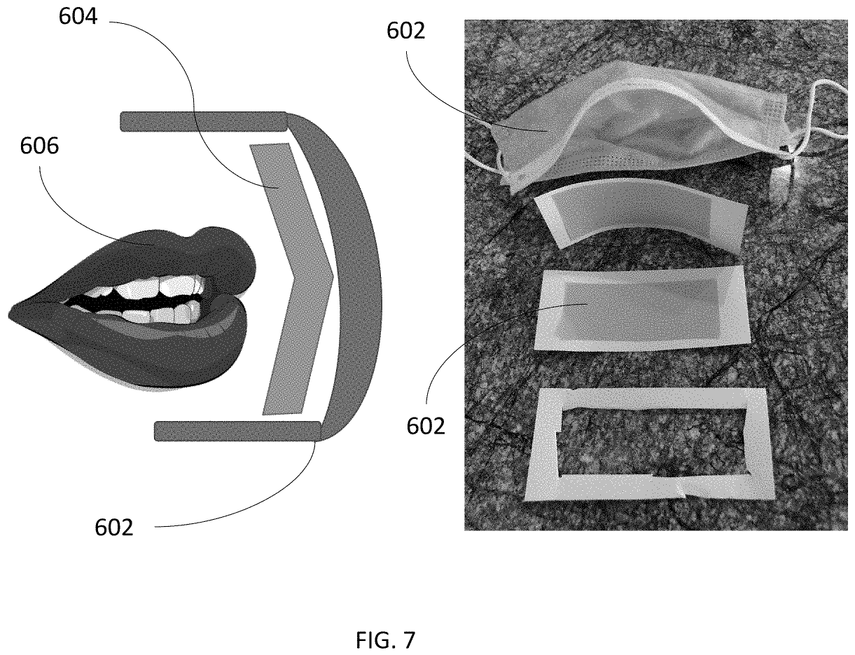

FIG. 7 depicts an article of the invention configured for use with a facemask 604 and the orientation of the mouth 606 of the wearer, the article, and the facemask. The article may be a silica gel carrier frame. The article may be pre-curved to allow space between the lips and nose of the user and the article to decrease contact of lips with the article. The article may be shipped folded without an embedded frame and the frame may be added after shipping to allow the article to hold its shape. The article may also be shipped with the embedded frame in place.

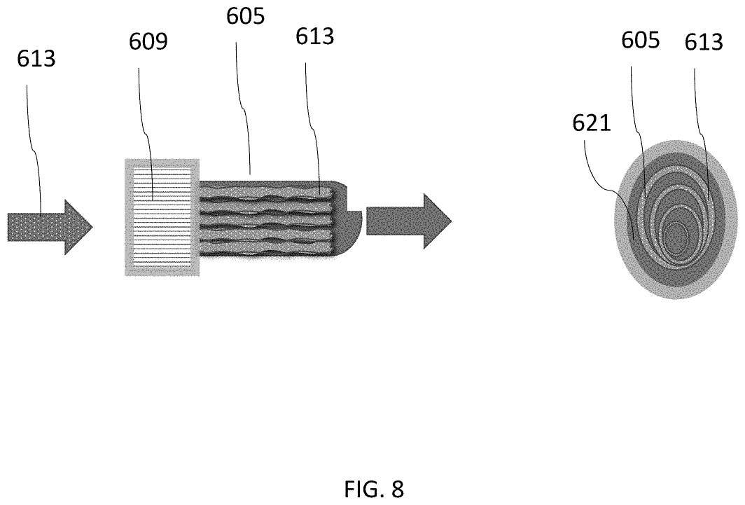

FIG. 8 depicts an aspect of the invention configured as a collector tube 605. The collector tube 605 comprises the adsorbent or adsorbent material and optionally an infectious agent binding material, for example a material comprising antibodies. The collector tube 605 comprises a mouth piece or nares sampling tube 609 at one end of the tube. Air, depicted as a blue arrows, comprising respiratory droplets which may comprise target microbes 613, depicted by as white dots, passes through the article and are collected by the collector tube 605. The air then exits the collection device reasonable free of respiratory droplets and viral particles. A cross-section 617 of the collector tube 605, depicts an exemplary system within the tube for collecting respiratory droplets. The collector tube is inserted as a loosely rolled tube with spacers separating layers of the absorbent or adsorbent material within the collector tube.

The collector tube 605 can be used for oral sampling methods and/or nasal sampling methods. The tube may be placed either in the mouth and/or nostrils and exhaled air is delivered into the collector tube that allows the exhalant to contact material that absorbs or adsorbs respiratory droplets and the contents of the respiratory droplets as the exhalant passes through the collector tube and out the collector tube exit port. The material may or may not be removed from the device prior to analyzing the sample. The collector tube may be mechanically fixed to the patient's oral or nasal cavity, for example with a cannula or by medical tape, or may be held in place by the user or a third party.

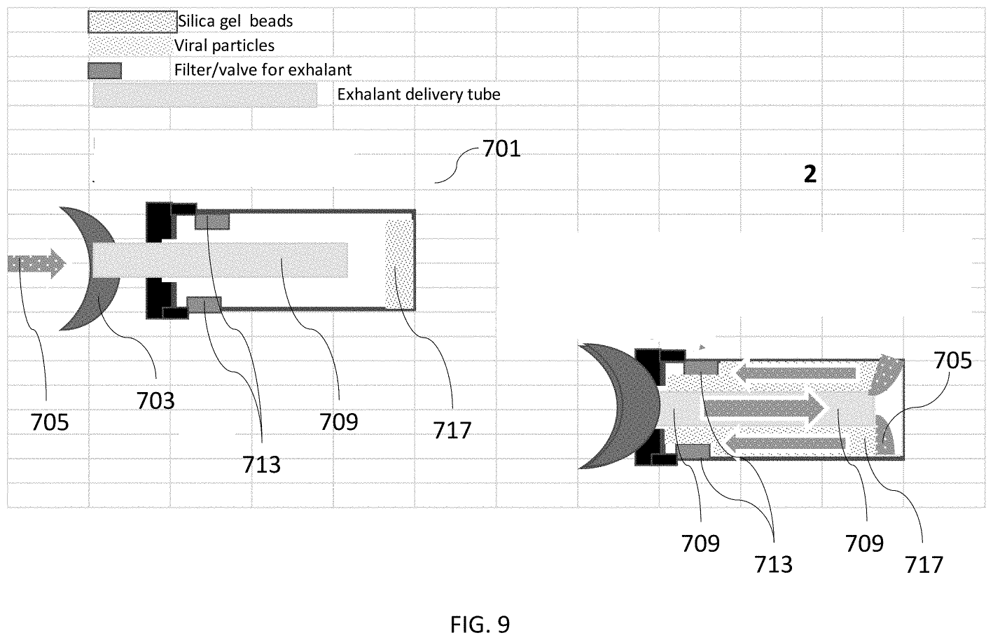

FIG. 9 depicts an article of the invention configured as a collector tube 701 with a mouthpiece 703 for oral collection. The mouthpiece 703 is connected to a delivery tube 709 that passes into the collector tube 701. Air, depicted by orange arrows, air comprising respiratory droplets comprising microbes 705, depicted as white dots, passes through the mouthpiece 703 into the delivery tube 709 and makes contact with the material capable of adsorbing or absorbing respiratory droplets 717. The material may be, for example, silica beads within the collector tube. In aspects of the invention, the air travels down the tube and may violently mix with the material 717, dispersing the material within the collector tube. The material 717 adsorbs and/or absorbs respiratory droplets from the air. The material 717 may comprise a color indicator that indicates adsorption and/or absorption of moisture in the air by the, for example, silica beads. The collector tube may further comprise at least one filter 713 that retains the material 717 within the collector tube while allowing air to leave through the filters 713. This is advantageous where the material is shaped as silica beads to prevent the beads from escaping the collector tube.

The mouthpiece 703 may be configured to be removable from the collector tube 701. The mouthpiece 703 may attach to the collector tube by any known means. For example, the mouthpiece may attach and detach from the collector tube through a screw type assembly or an air-tight fitted assembly. The mouthpiece 703 may be shaped to improve oral collection, for example, the mouthpiece may be flanged.

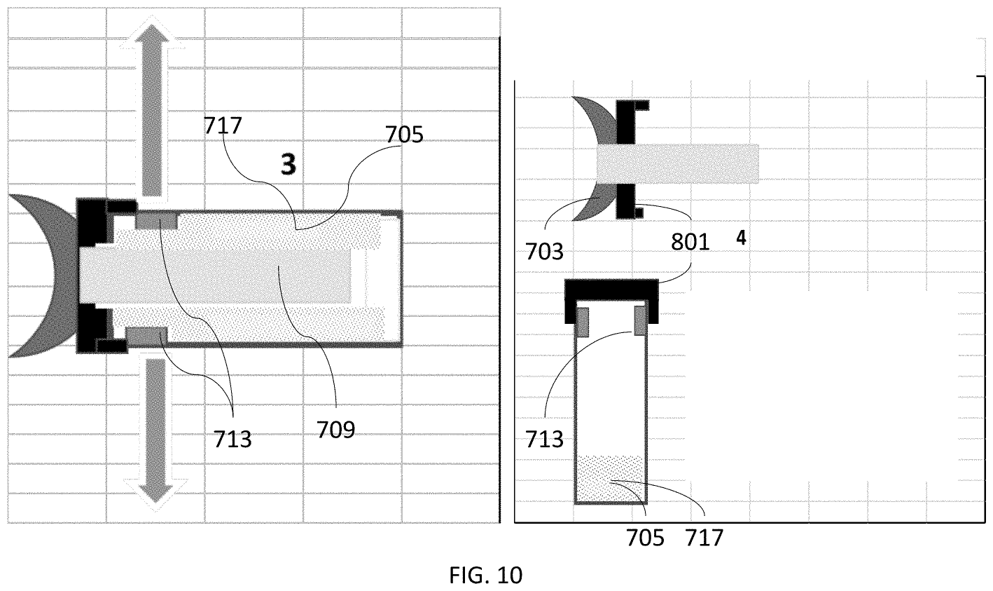

FIG. 10 depicts an article of the invention configured as a collector tube. Air, depicted by orange arrows, that has passed through the collector tube exits the collector tube through filters. The filters 713 retain the material 717 of the invention within the tube that has adsorbed or absorbed respiratory droplets from air that has passed through the mouthpiece 703 and delivery tube 709. Microbes within the respiratory droplets 605 collected by the material 717 is also collected by the material and retained within the collector tube. After air from a subject has passed through the collector device 701, the mouthpiece 703 may be removed and discarded. Caps 801 comprising the mouthpiece assembly or provided separately from the mouthpiece assembly may then be used to seal the end of the collector tube that held the mouthpiece. The caps 801 may extend over the filters 713. The adsorbent or absorbent material 717, adsorbed or absorbed respiratory droplets, and microbes 705 are retained within the collector tube. The collector rube 701 may then be sent for analysis. Advantageously, a buffer or any reagents may be added directly to the collector tube. The buffer or reagents may be added to the collector tube prior to sealing the collector tube and/or prior to shipping the collector tube. For example, the cap 801 for the collector tube may comprise a self-sealing syringe penetrable portion through which reagents may be introduced to the sealing collector tube.

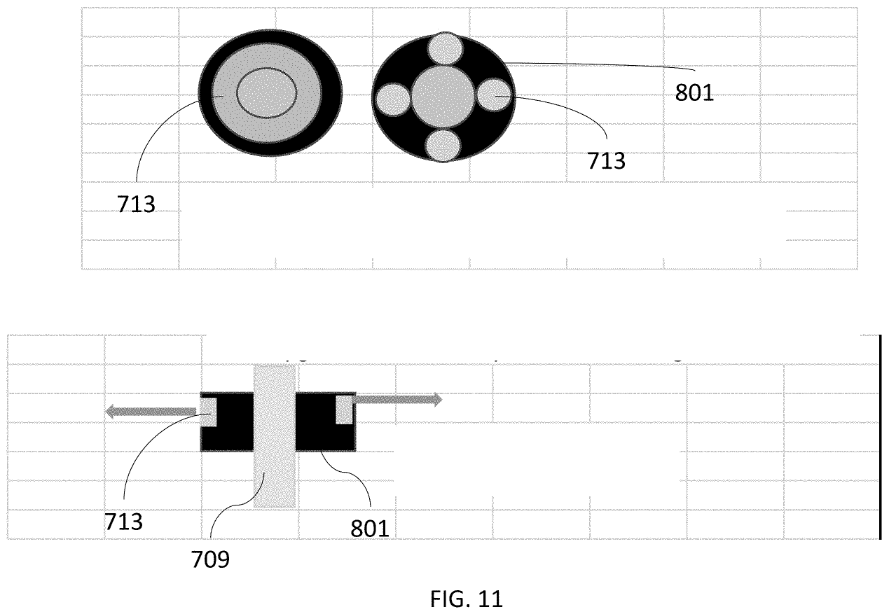

FIG. 11 depicts a cross-section of an article of the invention configured as a collector tube. The cap 801 fits over the filter 713 of the collector tube. The filter may be shaped around the entire circumference of the collector tube. The filter 713 may be shaped as separate filters positioned within the collector tube. The filter 713 may comprise a paper. The cap assembly 801 may fit over the delivery tube 709. The at least one filter 813 may be disposed within the cap apparatus, which allows air, depicted by orange arrows, to exit the collector tube. The filters 713 retain the adsorbant or absorbant material within the collector tube.

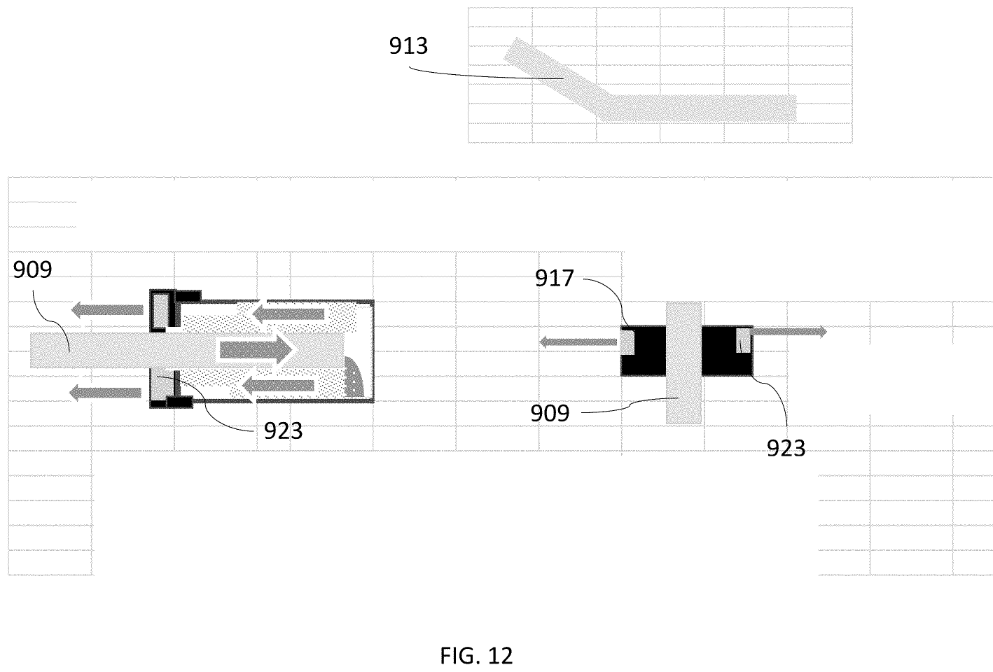

FIG. 12 depicts an aspect of the invention configured as a collector tube without a mouthpiece assembly. The delivery tube 909 of the collector tube allows for entry of air into the collector tube. The delivery tube may be bent 913 to control air entering the collector tube. The cap assembly 917 fits over the delivery tube 909, The at least one filter 923 is disposed within the cap apparatus, which allows air, depicted by orange arrows, to exit the collector tube. The filters 917 retain the material within the collector tube. The one or more filters 917 may be disposed in a lateral wall of the cap assembly and air leaving the collector tube may exit the side of the cap assembly through the filter. The diameter of the delivery tube 909 may be adjusted to control the speed at which air enters the collector tube. The speed at which air enters collector tube may be adjusted to adjust the impact of the air upon the material, and thereby the dispersal of material within the collector tube. The speed at which air enters the collector tube may also be adjusted to prevent loss of the material due to material sticking to the interior of the delivery tube 909. The diameter of the delivery tube 909 may also be adjusted for oral collection or nasal collection. For example, a larger diameter that a user can place their lips around may be used for oral collection and a smaller diameter to be inserted into the nostril of a subject may be used for nasal collection. Delivery tubes 909 for both oral and nasal collection may advantageously comprise a flexible material. The portion of the delivery tube 909 through which air is collected may also comprise an attachment, for example a firm tip or end may be used at the collection end of the delivery tube.

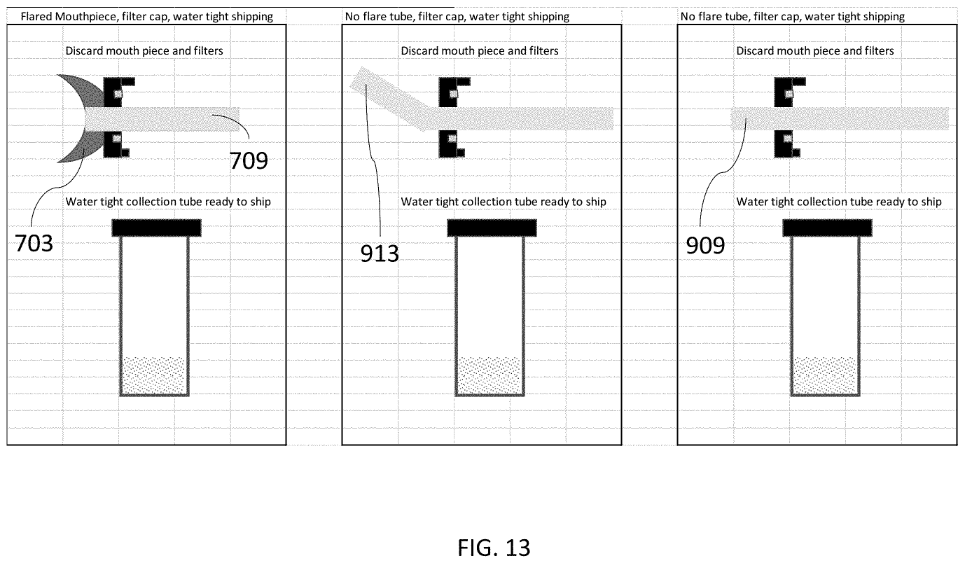

FIG. 13 depicts aspects of the invention configured as a collector tube. The collector tube may comprise a mouthpiece 703 detachably connected to a delivery tube 701. The delivery tube of the collector tube may provide the collection portion of the collector tube without the assistance of a mouthpiece. The collector tube may be bent 913 or may be straight 909. The cap assembly of each collector tube may be used to seal the collector tube. The seal may be water tight. The collector tube may then be shipped for analysis.

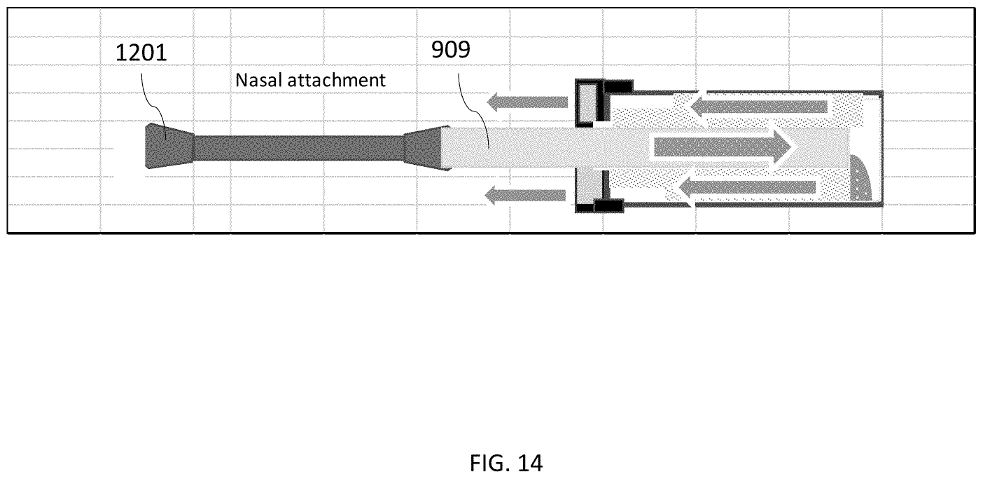

FIG. 14 depicts an aspect of the invention configured as a collector tube for nasal collection. The collector tube comprises a delivery tube 909 not connected to a mouthpiece. The collector tube is connected to a nasal attachment 1201. The diameter of the nasal attachment may be narrower than the delivery tube. Advantageously, the nasal attachment may be flexible. The collection end of the nasal attachment adapted to fit over one or both nostrils of the subject or may be adapted to be inserted into one or both nostrils of the subject.

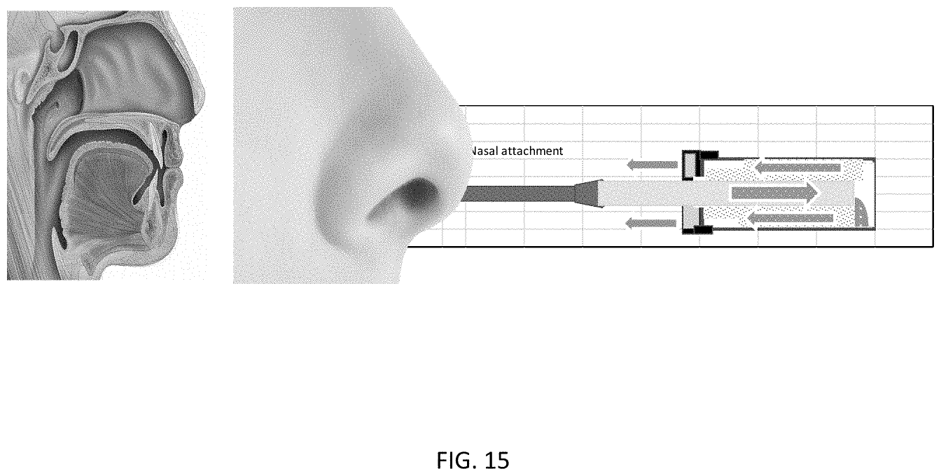

FIG. 15 depicts an aspect of the invention configured as a collector for nasal collection shown oriented with the nose of the patient. The collector tube is connected to a nasal attachment. The diameter of the nasal attachment allows for the delivery tube to enter the left nostril of the subject. The subject then exhales through the left nostril permitting collection of respiratory droplets from the nose.

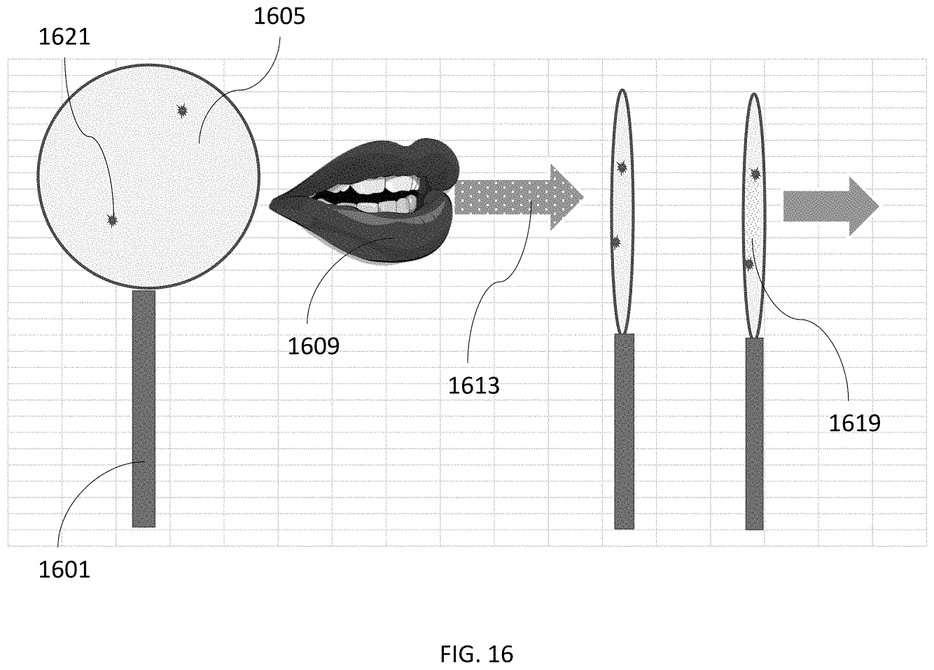

FIG. 16 depicts an aspect of the invention configured as a hand-held or “lollipop” collection device. The adsorbent or absorbent material of the device 1605 is configured with a handle 1601 that holds the material in place. The user expels air, depicted by orange arrows, through their mouth 1605 or nose held in front of the hygroscopic material. Respiratory droplets to be sampled and analyzed 1613 are passed through the article. The respiratory droplets are collected 1619 by the material and may then be analyzed. For example, the device may be placed into a sealed container, such as a plastic container, and shipped to a laboratory for testing.

Hand-held aspects of the invention may be held in front of the mouth by the user similar to a lollipop or “blow pop”, which may be advantageous for testing of children. The article may further be configured to comprise a bead 1621. Once exposed to or saturated with moisture, the bead 1621 may change color to indicate saturation.

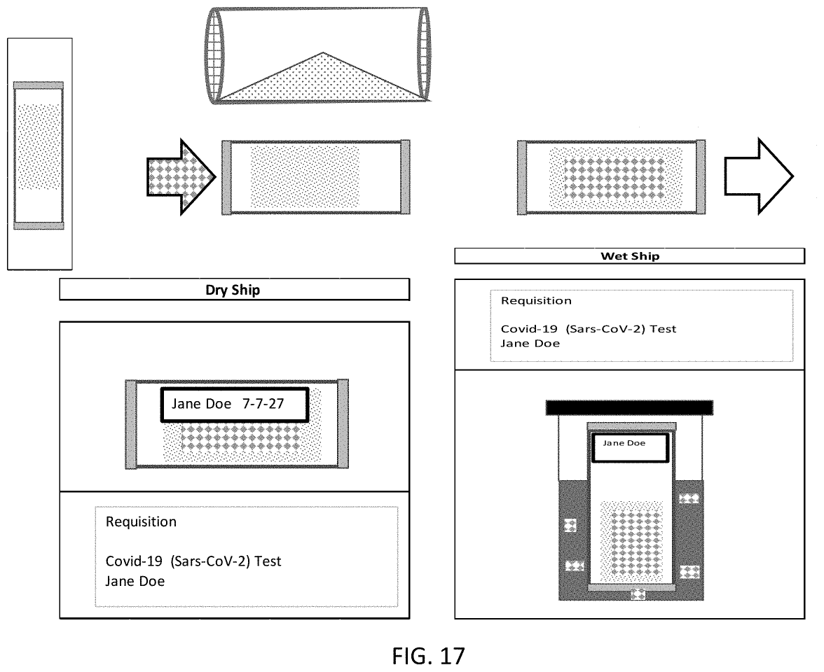

FIG. 17 depicts an aspect of the invention configured as a “soda straw” collection device. The device comprises a cylindrical tube. The tube may advantageously be 0.5-0.75 inches in diameter. The device comprises the material, such as a hygroscopic bead within the cylinder. The device may preferable comprise 2-3 grams of material for a 0.5-0.75 inch cylinder. The cylinder is sealed at both ends by a porous mesh that comprises beads. The beads of the mesh may be 500-750 μM beads or approximately 1 mm beads. The beads may be greater than 1 mm and/or may be optimized as needed. Force of breath from a user mixes the material with exhalant within the straw. The device advantageously can be used when the user tilts their head slight downward. The device can be shipped as is or may be inserted into a secondary tube, such as a straw. A stabilizing extraction buffer can be used to initiate analysis. The device may be individually wrapped in a paper sleeve, similar to the packaging of soda straws. The user may un-wrap the paper sleep, remove the device, and place either end of the device to their mouth or nostril. For nasal use, the device may be fitted with a nasal extension tube. The user exhales and respiratory droplets comprising particles to be analyzed are collected by the hygroscopic material in the cylinder. Post-collection, the device is packed “dry” or placed in a container for shipping. The container for shipping may be shipped “wet” by pre-loading the container with a target extraction buffer so that the sample is immediately ready to be analyzed upon on receipt by the analytic laboratory. The straw may be a wide straw, a standard ¾ inch straw, or a narrower straw. Narrower straws may be advantageous when the user is a child. Advantageously “straw” embodiments allow for easy handling and provide space for affixing an identification label.

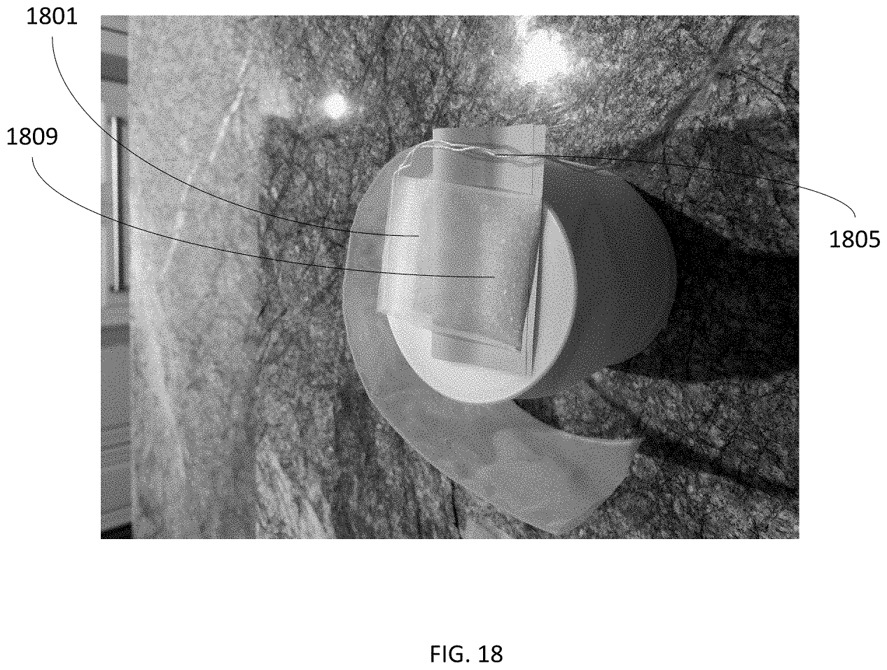

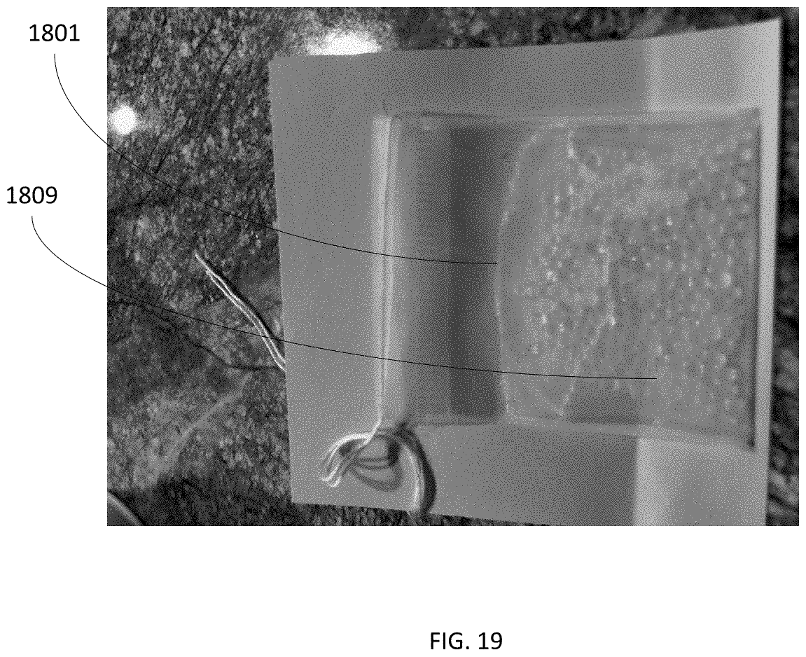

FIG. 18 and FIG. 19 depict “tea-bag” shaped article of the advice. The adsorbent or absorbent material of the article may be incorporated into the pouch shape of the tea bag 1801. For example, the “tea-bag” article may comprise food-grade mesh bags, for example nylon tea bags. The “tea-bag” article may comprise a mesh within the tea bag or at the surface of the tea bag sufficient to contain beads 1809 that comprise the adsorbent or absorbent material. The mesh may be about 160 nm. Respiratory air in breathe from a user may easily pass through the article, with respiratory droplets and the contents of the respiratory droplets being captured by the beads 1809 of the “tea bag” article. The “tea-bag” article may comprise a string 1805 for the user to hold the article and exhale onto the pouch shape of the tea bag. The “tea-bag” article may be affixed to clothing of the user, for example affixed by a pin and worn like a badge by the user. The “tea-bag” article may comprise colorimetric beads that change color to indicate when the “tea-bag” is saturated with moisture.

The article may come in a package together with instructions that instruct the user as to the distance at which to hold the article in place. The article may come in a package together with instructions that instruct the user as to the length of time for which to hold the article in place. The “tea-bag” article may be shipped for analysis dry, or may be packaged together with an extraction buffer or stabilizing buffer, for example in a sealed vial. The tea-bag size may be about 2×2.4 inches or about 2×2 inches, which advantageously lends itself to being capable of being placed on the inner surface of a facial covering, for example a surgical mask.

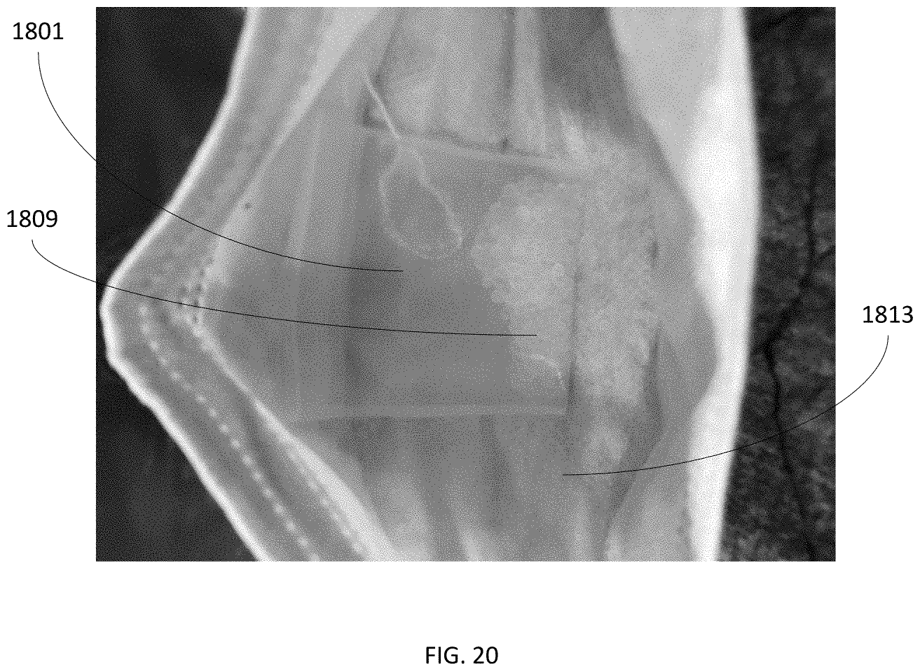

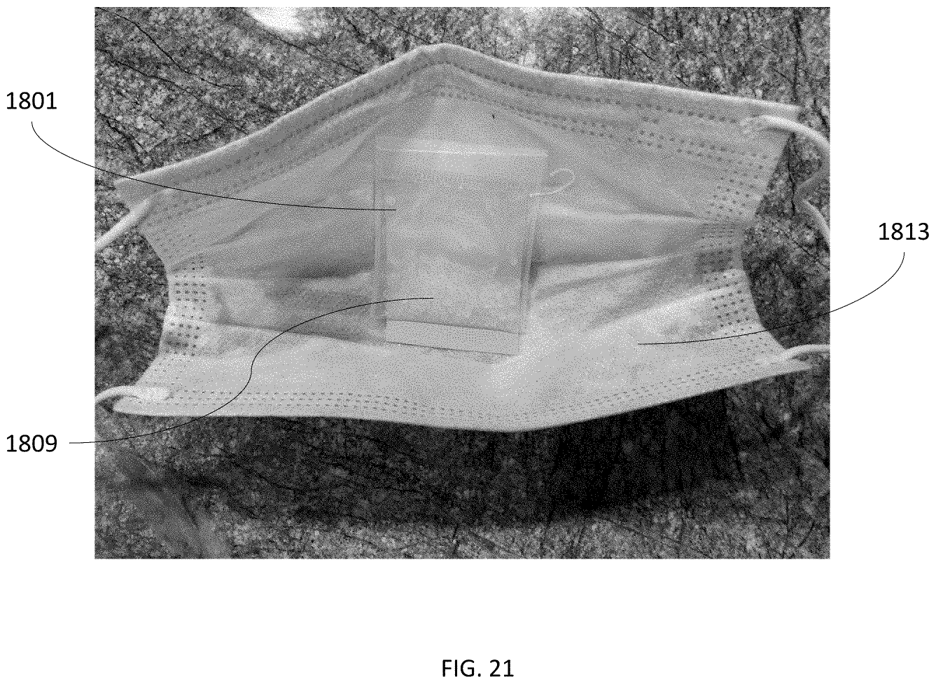

FIG. 20 and FIG. 21 depict “tea-bag” article of the invention together with a facial covering. Using a tea-bag article together with a facial covering 1813 ensures that the beads are in a high moisture atmosphere, so mixing of the beads, as they settle, is mitigated. The “tea-bag” article 1801 and/or beads 1809 are advantageously food grade and tasteless, which prevents toxicity of the article. The “tea-bag” article 1801 may be incorporated into the surgical mask 1813 by any known method. For example, the “tea-bag” article may comprise an adhesive at the top corners of the “tea-bag” article to position the “tea-bag” article inside the facial oral and/or nasal covering. A wire may be added to further contour the device.

Aspects of the invention may comprise a mesh at the point of contact between the device and exhaled air. The mesh may comprise beads. Beads are preferably greater than 0.75 mm in diameter in order to not significantly impede breath. The mesh advantageously serves to protect the user from inhaling the material or the material from otherwise escaping the device while still allowing air to pass through the device, The mesh can be welded, for example ultrasonically welded, or otherwise adhered to the device. The mesh may be shaped to snap-fit onto one or more ends of the device. The mesh and beads comprising the mesh may be commercially available. The mesh and other aspects of the device may be sterilized, for example using UV sterilized or chemically sterilized. Aspects that require contact with the mouth or nose of the subject advantageously may be sterilized using a process that avoids imparting a taste on the device.

The article may also be shaped to filter circulating air. For example, the article may be shaped to filter air in an air purification system or an air conditioning system, such as a humidifier, dehumidifier, air exchanger, air cleaner, or HVAC system. The article may be shaped to filter air passing through a vent.

Heating, ventilation, and/or air conditioning (HVAC) systems control the temperature within a building or other structure. HVAC systems may include boiler systems, radiant heating systems, electric heating systems, a system of ductwork and air vents, and one or more HVAC controllers. The one or more HVAC components may include a furnace, a heat pump, an electric heat pump, a geothermal heat pump, an electric heating unit, an air conditioning unit, a humidifier, a dehumidifier, an air exchanger, an air cleaner, and/or the like. HVAC systems typically include an air filter to help remove dust and other pollutants from within the building and to protect the HVAC equipment from dust buildup which may negatively impact system performance. The article of the present invention may be provided as a filter for an HVAC system, or may be provided as part of an existing filter for an HVAC system. HVAC systems are described in U.S. Pat. No. 10,119,718, herein incorporated by reference in its entirety.

The invention also provides methods of analyzing the presence of microbes, such as viral particles using the articles and devices of the invention. The method may analyze respiratory droplets by passing air through the article comprising an absorbent or adsorbent material, wherein the material collects respiratory droplets in the air, recovering the contents of the droplets from the material, and analyzing the contents of the droplets.

Advantageously, articles, devices, and methods of the present invention allow for the contents of respiratory droplets to be recovered from the article after collection to be further analyzed. The method of recovery in the case of an adsorbent material may elution, for example by the addition of an aqueous buffer, for example but not limited to saline or phosphate-buffered saline, onto the adsorbent material. For some absorbent materials, addition of an aqueous buffer may suffice to extract the droplets. For most absorbent materials, additional treatment will be required to recover the contents of the respiratory droplets. These treatments will vary depending on the characteristics of the absorbent material used, and could include for example centrifugation, temperature modulation, or the addition of solvents or other chemicals or molecules that serve to liberate the absorbed droplets from the absorbent material. Upon recovery, the droplets will be coalesced into an aqueous phase for subsequent analysis.

Analyses of the contents of the respiratory droplets may comprise any known method of analysis, for example any method for detecting the presence of identity of a virus. The analysis may be used to detect any target analyte. The target analyte refers to the substance in the sample that will be captured and isolated. The target analyte may be inorganic (e.g., a metal, a cyanide or cyanate, a salt, etc.) or organic chemicals, macromolecules (chitin, peptidoglycan, carbohydrates, proteins, nucleic acids, lipids, etc.,), bacteria, fungi, a cell (such as a cancer cell, a white blood cell a virally infected cell, or a fetal cell circulating in maternal circulation), a virus, a nucleic acid (e.g., DNA or RNA), a receptor, a ligand, a hormone, a drug, a chemical substance, or any molecule known in the art. In preferred aspects of the invention, the analyte is not a volatile organic compound.

For example, the analysis may comprise DNA or RNA amplification, such as PCR, nucleic acid sequencing, such as next generation sequencing (NGS) and/or droplet-based sequencing, anti-body based analysis, an immunoassay, or any combination of analytic techniques and methods.

Nucleic acid template molecules (e.g., DNA or RNA) can be isolated from a biological sample in the respiratory droplet containing a variety of other components, such as proteins, lipids, and non-template nucleic acids. Nucleic acid template molecules can be obtained from any cellular material, obtained from animal, plant, bacterium, fungus, or any other cellular organism. Biological samples for use in the present invention also include viral particles or preparations. Nucleic acid template molecules can also be isolated from cultured cells, such as a primary cell culture or cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA from the freezer.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, N.Y. 2,028 pages (2012); or as described in U.S. Pat. Nos. 7,957,913; 7,776,616; 5,234,809; U.S. Pub. 2010/0285578; and U.S. Pub. 2002/0190663.

Nucleic acid from a sample may optionally be fragmented or sheared to a desired length, using a variety of mechanical, chemical, and/or enzymatic methods. DNA may be randomly sheared via sonication using, for example, an ultrasonicator sold by Covaris (Woburn, Mass.), brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. Generally, individual nucleic acid template molecules can be from about 2 kb bases to about 40 kb, Nucleic acid molecules may be single-stranded, double-stranded, or double stranded with single-stranded regions (for example, stem- and loop-structures).

A biological sample may be lysed, homogenized, or fractionated in the presence of a detergent or surfactant as needed. Suitable detergents may include an ionic detergent (e.g., sodium dodecyl sulfate or N-lauroylsarcosine) or a nonionic detergent (such as the polysorbate 80 sold under the trademark TWEEN by Uniqema Americas (Paterson, N.J.) or C14H22O(C2H4)n, known as TRITON X-100).

A target may be analyzed by a multitude of existing technologies, such as nuclear magnetic resonance (NMR), miniature NMR Polymerase Chain Reaction (PCR), mass spectrometry, fluorescent labeling and visualization using microscopic observation, fluorescent in situ hybridization (FISH), growth-based antibiotic sensitivity tests, and variety of other methods that may be conducted with purified target without significant contamination from other sample components. Analysis using NMR is described in U.S. Pub. 2011/0262925, herein incorporated by reference in its entirety.

PCR may be used as described or any other amplification reaction may be performed. Amplification refers to production of additional copies of a nucleic acid sequence and is generally carried out using polymerase chain reaction (PCR) or other technologies known in the art. The amplification reaction may be any amplification reaction known in the art that amplifies nucleic acid molecules such as PCR (e.g., nested PCR, PCR-single strand conformation polymorphism, ligase chain reaction, strand displacement amplification and restriction fragments length polymorphism, transcription based amplification system, rolling circle amplification, and hyper-branched rolling circle amplification, quantitative PCR, quantitative fluorescent PCR (QF-PCR), multiplex fluorescent PCR (MF-PCR), real time PCR (RTPCR), restriction fragment length polymorphism PCR). See U.S. Pat. Nos. 5,242,794; 5,494,810; 4,988,617; 6,582,938; 4,683,195; and 4,683,202, hereby incorporated by reference. Primers for PCR, sequencing, and other methods can be prepared by cloning, direct chemical synthesis, and other methods known in the art. Primers can also be obtained from commercial sources such as Eurofins MWG Operon (Huntsville, Ala.) or Life Technologies (Carlsbad, Calif.).

Alternatively, an isothermal method of amplification, e.g. rolling circle amplification (RCA) or loop-mediated isothermal amplification (LAMP), may be performed.

Amplification adapters may be attached to the fragmented nucleic acid. Adapters may be commercially obtained, such as from Integrated DNA Technologies (Coralville, Iowa). The adapter sequences may be attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase, available commercially from New England Biolabs (Ipswich, Mass.). Methods for using ligases are well known in the art. The polymerase may be any enzyme capable of adding nucleotides to the 3′ and the 5′ terminus of template nucleic acid molecules.

Analysis may also involve attaching the bar code sequences to the template nucleic acids e.g., for barcode PCR. A bar code may be attached to each fragment. A plurality of bar codes, e.g., two bar codes, may be attached to each fragment. A bar code sequence generally includes certain features that make the sequence useful in sequencing reactions. For example the bar code sequences are designed to have minimal or no homo-polymer regions, i.e., 2 or more of the same base in a row such as AA or CCC, within the bar code sequence. The bar code sequences are also designed so that they are at least one edit distance away from the base addition order when performing base-by-base sequencing, ensuring that the first and last base do not match the expected bases of the sequence.

The bar code sequences are designed such that each sequence is correlated to a particular portion of nucleic acid, allowing sequence reads to be correlated back to the portion from which they came. Methods of designing sets of bar code sequences are shown for example in U.S. Pat. No. 6,235,475, the contents of which are incorporated by reference herein in their entirety. Since the bar code sequence is sequenced along with the template nucleic acid, the oligonucleotide length should be of minimal length so as to permit the longest read from the template nucleic acid attached. Generally, the bar code sequences are spaced from the template nucleic acid molecule by at least one base (minimizes homo-polymeric combinations). The bar code sequences are attached to the template nucleic acid molecule, e.g., with an enzyme. The enzyme may be a ligase or a polymerase, as discussed below. Attaching bar code sequences to nucleic acid templates is shown in U.S. Pub. 2008/0081330 and U.S. Pub. 2011/0301042, the contents of which are incorporated by reference herein in its entirety. Methods for designing sets of bar code sequences and other methods for attaching bar code sequences are shown in U.S. Pat. Nos. 7,544,473; 7,537,897; 7,393,665; 6,352,828; 6,172,218; 6,172,214; 6,150,516; 6,138,077; 5,863,722; 5,846,719; 5,695,934; and 5,604,097, each incorporated by reference.

After any processing steps (e.g., obtaining, isolating, fragmenting, amplification, or barcoding), nucleic acid can be sequenced.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

An advantage of the current invention is its ability to collect the respiratory material in a manner that enables subsequent assessment of active infectious particles in the material. Thus, when respiratory droplets are sampled using the articles and methods described herein, the recovered material may be analyzed functionally for the presence of active virus or bacterial particles. For example, active virus can be assessed by placing the recovered material onto cells in culture to determine if the virus in the recovered respiratory material is capable of self-propagation. Active bacteria can be assessed, for example, by placing the recovered material onto agar plates infused with the appropriate growth medium. Viable infectious agents may be assessed quantitatively or qualitatively using any number of methods known to those versed in the art. This contrasts with other methods for harvesting respiratory droplets, which often compromise the viability of any active infectious particles present in the droplets.

### EXAMPLES

### Example 1

Articles of the invention comprising silica gel beads were tested for their absorption and adsorption of moisture.

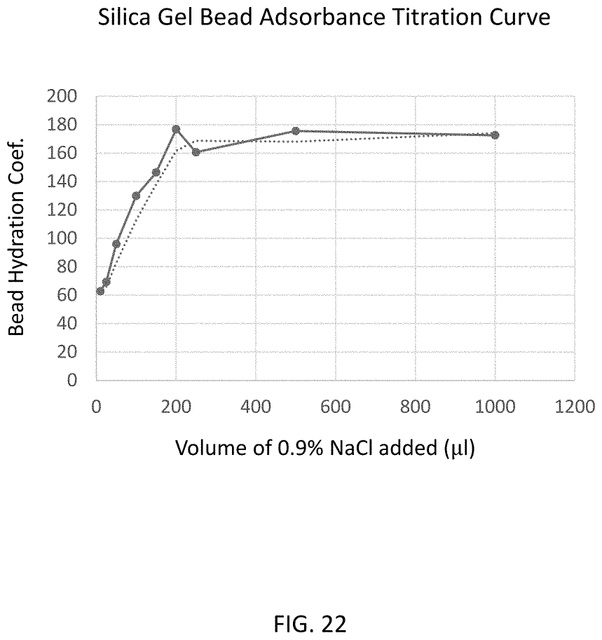

FIG. 22 depicts results of adsorbance of saline by silica gel beads. Saline (0.9% Na Cl) was dripped in the volumes shown onto 350 mg of silica gel beads. The beads had a size range 200 um-400 um and contained <0.3% methyl violet dye which served as a colorimetric indicator of hydration.

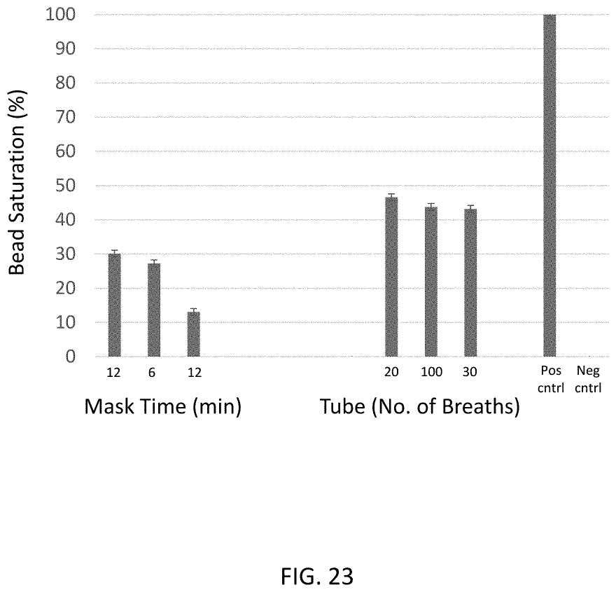

FIG. 23 depicts adsorption of exhalant vapors from human subjects onto silica gel beads in mask and tube embodiments described above. Human subjects were provided two form factors containing 350 mg of silica gel beads with a size range of 200 um-400 um.

Results for surgical masks with a nylon mesh teabag embodiment are shown to the left. Silica gel beads of a size range between 0.2-0.4 mm (200-400 um), with a methyl violet colorimetric indicator of hydration, were enclosed within a nylon mesh teabag embodiment, and the teabag affixed to the interior of a standard surgical face mask so that it was positioned between the mask and the mouth. Subjects were asked to breathe as they would normally for either 6 or 12 minutes as shown. Recovery of vapor from the breath was quantified as bead saturation, based on the colorimetric change.

Results for blow tube form embodiments are shown to the right. Silica gel beads with the same characteristics as above were placed inside a 4-inch-long tube with a diameter of 1 inch. The tube at one end has a removable cap, through which the subject blows, and a nylon mesh covering the other end of the tube through which the exhalant exits, allowing for air and vapors from the exhalant to flow freely through the tube while retaining the silica gel beads within the tube. The cap was removed, and subjects were asked to exhale into the tube for the indicated number of breaths. Subsequently the silica gel beads were analyzed for the recovery of vapor from the exhalant, quantified as bead saturation based on the colorimetric change. Positive control was 350 mg of silica gel beads incubated with 400 ul of 0.9% saline, while negative controls were untreated silica gel beads.

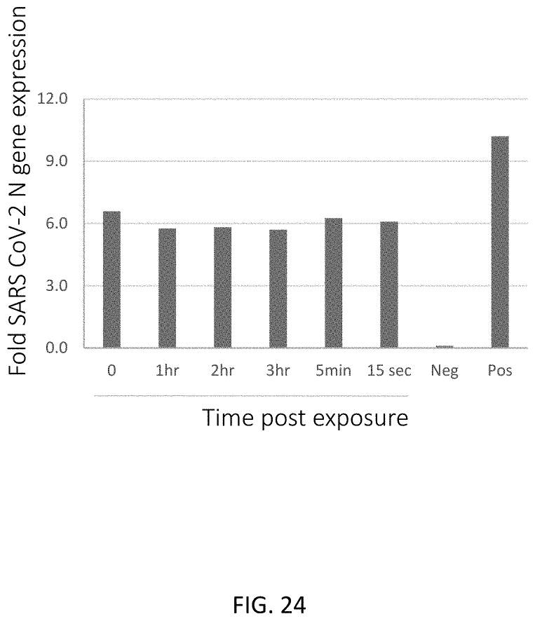

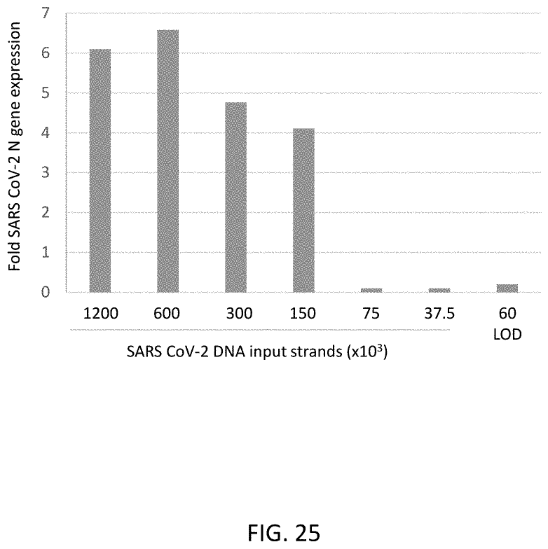

FIG. 24 and FIG. 25 depict results for the recovery of aerosolized droplets of SARS-CoV-2 nucleic acid from silica gel beads. A stock solution of 1.2×10⁶ DNA molecules of inactivated SARS-CoV-2 DNA, in a total volume of 111 ul was used for recovery assays that measured both stability (A) and limiting dilution (B). Aerosolized inactivated SARS-CoV-2 virus droplets of size ranging from 1.9 μm and 3.7 μm were exposed to a nylon mesh teabag containing 0.943 g silica gel beads. Beads were isolated and analyzed by RT-PCR using the EXAS1002 test for SARS-CoV-2 (COVID19) nucleocapsid (N) gene, that has EUA approval by the FDA. (A) 60,000 strands/ml of N-gene Sars-CoV-2 DNA was aerosolized onto of silica gel beads and incubated at ambient temperature for indicated time points before bead elution and RT-PCR analysis. The negative (Neg) control were beads exposed to 1 mL aerosolized 0.9% saline solution. The positive (Pos) control were beads exposed to 54,000 strands/ml non-aerosolized N-geneSars-CoV-2 DNA. (B) Decreasing quantities of N-geneSars-CoV-2 DNA were aerosolized and 10 minutes after exposure, gel beads were eluted and analyzed by RT-PCR using EXAS1002 test. The Limit of Detection of the test was that of the EXAS1002 Assay.

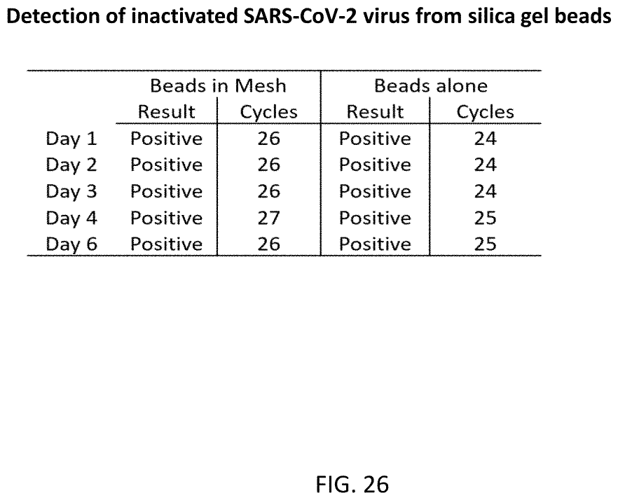

FIG. 26 depicts results for SARS-CoV-2 detection from the silica beads. Silica gel beads were enclosed in a nylon mesh sack (120 mm mesh), approximately 2×2 by 0.25 inches, in accordance with the standard tea bag embodiments described above. Each nylon mesh enclosure contained 0.8 grams of silica gel beads that were on average 200 um in diameter which provided a maximal adsorption capacity of 400 ul per enmasked silica gel bead nylon mesh bag. The tea bag was applied to the inner layer (oral-nasal facing) surface of a standard commercial three-ply facial mask generally used to prevent the spread of SARS-Cov-2 virus. 100,000 copies/ml of inactivated SARS-Cov-2 virus was suspended in nuclease free phosphate buffered saline and aerosolized. 35,000 viral copies of aerosolized inactivated SARS-Cov2 virus were delivered to the silica gel beads in the mesh bag. To control for any mesh effect on PCR, the same aerosol was applied to beads alone in a weighing boat. The saturated beads were allowed to sit at ambient temperature for varying times. Beads were isolated and analyzed by RT-PCR using the EXAS1002 test for SARS-CoV-2 (COVID19) nucleocapsid (N) gene. A positive result required a PCR cycle number of <40 and a negative result a cycle number greater than or equal to 40. Inactivated SARS-CoV-2 was detected on all six days on both beads in mesh and beads alone.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. An article for sampling respiratory droplets, the article comprising:
a material capable of adsorbing or absorbing respiratory droplets such that the contents of said respiratory droplets can be recovered for analysis.

2. The article of claim 1, wherein the respiratory droplets may be adsorbed or absorbed from one or more of the group consisting of moisture, exhaled air, circulating air, and ambient air.

3. The article of claim 1, wherein the material is a hygroscopic material.

4. The article of claim 3, wherein the hygroscopic material is capable of adsorbing respiratory droplets.

5. The method of claim 4, wherein the contents of the respiratory droplets may be recovered by elution.

6. The article of claim 1, wherein the contents of the respiratory droplets can be recovered for the analysis of microbes.

7. The article of claim 6, wherein the microbes are viral or bacterial.

8. The article of claim 3, wherein the material is selected from at least one of a desiccant or a superabsorbent polymer.

8. A device comprising the article of claim 1.

9. The device of claim 8, wherein the device is a face mask or a filter for circulating air or ambient air.

10. The device of claim 9, wherein the face mask is a surgical mask and the filter for circulating air or ambient air is a filter for an HVAC system, a filter for a vent, or a filter for a fan.

11. The device of claim 8, wherein the device is collection device for exhaled air.

12. The device of claim 11, wherein the device is an oral collector tube, a nasal collector tube, a hand-held lollipop device, a hand-held tea-bag device, a surface-resting device, a straw, or an insert for a straw.

13. A method for sampling respiratory droplets, the method comprising:
contacting with respiratory droplets a material capable of adsorbing or absorbing respiratory droplets;
recovering the contents of the respiratory droplets from the material;
analyzing the recovered contents of the respiratory droplets.

14. The method of claim 13, wherein the respiratory droplets may be adsorbed or absorbed from one or more of the group consisting of moisture, exhaled air, circulating air, and ambient air.

15. The method of claim 13, wherein the material is a hygroscopic material.

16. The method of claim 15, wherein the hygroscopic material is capable of adsorbing respiratory droplets.

17. The method of claim 16, wherein the contents of the respiratory droplets may be recovered by elution.

18. The method of claim 13, wherein the material part of a device.

19. The method of claim 18, wherein the device is a face mask, a filter for circulating air, a filter for ambient air, or a collection device for exhaled air.

20. The device of claim 19, wherein the device is a surgical mask, a filter for an HVAC system, a filter for a vent, or a filter for a fan. an oral collector tube, a nasal collector tube, a hand-held lollipop device, a hand-held tea-bag device, a surface-resting device, a straw, or an insert for a straw.
